(19) United States
(12) Patent Application Publication
(10) Pub. No.: US 2025/0009456 A1
(43) Pub. Date: Jan. 9, 2025

(54) MAGNETIC-FIELD GENERATING DEVICE FOR PRECISION PROCEDURE

(71) Applicant: IMSYSTEM CO.,LTD., Daegu (KR)

(72) Inventors: Jin Young KIM, Daegu (KR); Sung Woong JEON, Busan (KR); Eun Hee KIM, Daegu (KR)

(21) Appl. No.: 18/706,497

(22) PCT Filed: Dec. 15, 2021
(86) PCT No.: PCT/KR2021/019027

§ 371 Date: May 1, 2024

(30) Foreign Application Priority Data
Nov. 2, 2021 (KR) 10-2021-0148426

(51) Int. Cl. A61B 34/00; A61B 34/30

(52) U.S. Cl. CPC A61B 34/73; A61B 2034/303; A61B 2034/733

(57) ABSTRACT

The present invention relates to a magnetic-field generating device for a precision procedure and, more specifically, to a magnetic-field generating device for a precision procedure, in which, by extending a magnetic field generated from a magnetic coil to the upper side to extend the area of the magnetic field, a procedural robot that is inserted into a patient's body can be safely and precisely moved up to a target position by using the magnetic field. The magnetic-field generating device for a precision procedure, according to an embodiment of the present invention for solving the problem described above, comprises: a moving unit configured to be movable; a first magnetic-field generating unit installed in a moving enclosure; a second magnetic-field generating unit that generates a magnetic field so as to overlap the area of the magnetic field generated by the first magnetic-field generating unit.

### TECHNICAL FIELD

The present invention relates to a magnetic field generating device for a precision procedure, and more specifically, to a magnetic field generating device for a precision procedure, which may safely and precisely move a procedural robot (hereinafter referred to as a “micro magnetic robot”) inserted into a patient's body to a target position using a magnetic field by extending the magnetic field generated from a magnetic coil upward to extend an area of the magnetic field.

### BACKGROUND ART

Various attempts for performing vascular procedures, such as cardiac arrhythmia treatment and occlusive vascular treatment, or effectively delivering drugs in a blood vessel after inserting a microrobot into a patient's body and then moving the robot to a lesion location using an external magnetic field are continuously being made.

The key to the success of such a procedure method is to precisely control the microrobot in complex internal organs of the human body, especially blood vessels, and to achieve this, an effective magnetic driving system using an external magnetic field should be established. However, conventional magnetic driving systems are very large in size and devices are heavy and thus occupy a significant portion of a surgical bed or an operating room, resulting in significant space limitations and difficulties in precisely controlling the microrobot.

Therefore, as one of the technologies for controlling the movement of microrobots, a surgical bed incorporating a magnetic field control system and an imaging system was disclosed in Korean Patent No. 10-1720032.

The above technology includes a magnetic induction device provided under the bed to induce a magnetic field and the magnetic induction device is configured so that a plurality of magnetic induction coils are radially disposed to be spaced a certain distance from a reference point.

However, in the above technology, since a magnetic field generated from the magnetic induction coil interferes with a magnetic field generated from an adjacent magnetic induction coil to cancel the generated magnetic field, there is a problem that a lot of power is consumed to generate a magnetic field suitable for the movement control of a capsule robot located inside the human body.

In addition, a magnetic driving system and a microrobot control method using the same were disclosed in Korean Patent No. 10-2289065.

The above technology includes a first magnetic field generating unit, a second magnetic field generating unit disposed to face the first magnetic field generating unit with an operating area interposed therebetween, and a movement module for moving the first magnetic field generating unit and the second magnetic field generating unit, in which the movement module includes a body having a support shaft, a rotating arm coupled to the support shaft and rotatable about the support shaft, a pair of connecting arms each mounted on the tip of the rotating arm and rotatable about a first axis, a first support arm mounted on the tip of any one of the connecting arms, rotatable about a second axis parallel to the first axis with respect to the connecting arm, and supporting the first magnetic field generating unit, and a second support arm mounted at the tip of the other connecting arm, rotatable about a third axis parallel to the first axis with respect to the connecting arm, and supporting the second magnetic field generating unit.

However, in the above technology, although the magnetic field may be concentrated on the human body only when the first magnetic field generating unit and the second magnetic field generating unit are disposed to face each other, since the first magnetic field generating unit and the second magnetic field generating unit are configured to rotate about the axes, there is a problem in that it is difficult to arrange the first magnetic field generating unit and the second magnetic field generating unit to face each other.

### DISCLOSURE

### Technical Problem

The present invention was made to solve the above problems of the related art and is directed to providing a magnetic field generating device for a precision procedure, which may generate a uniform and strong magnetic field in a three-dimensional space by configuring a second magnetic field generating unit to further extend a magnetic field generated from a first magnetic field generating unit disposed under a patient upward.

In addition, the present invention is directed to providing a magnetic field generating device for a precision procedure, which may prevent a malfunction of a system due to heating generated from a core and minimize damage to a device due to overheating and overcurrent.

### Technical Solution

A magnetic field generating device for a precision procedure according to one embodiment of the present invention includes a moving unit configured to be movable, a first magnetic field generating unit installed on the moving unit, and a second magnetic field generating unit configured to generate a magnetic field to overlap a magnetic field area generated by the first magnetic field generating unit.

The moving unit includes an accommodating case in which the first magnetic field generating unit is installed, a driving module driven according to a driving control signal, and a control module configured to provide the driving control signal to the driving module based on received location information.

The control module is configured to detect a temperature value and an applied current value of the first magnetic field generating unit or the second magnetic field generating unit and control to interrupt the current applied to the first magnetic field generating unit or the second magnetic field generating unit on the detected temperature value and current value.

The first magnetic field generating unit is composed of a magnetic coil including a core, and a coil wound around the core.

The core may be configured to be cooled by one or more cooling methods selected from air cooling and water cooling.

The magnetic coils is configured as a plurality of magnetic coils at predetermined intervals.

The magnetic coil is shielded by a magnetic field shielding material.

The second magnetic field generating unit includes a lower coil disposed under the first magnetic field generating unit, and an upper coil disposed above the first magnetic field generating unit, wherein the lower coil and the upper coil are each formed in a donut shape.

The magnetic field generating device for a precision procedure includes a vertical frame installed upright on the moving unit, and a horizontal frame disposed perpendicular to the vertical frame, and the upper coil is installed on the horizontal frame.

The second magnetic field generating unit may be formed of a Helmholtz coil.

### Advantageous Effects

According to the present invention, since a magnetic field generated from a first magnetic field generating unit is concentrated in an effective space by a magnetic field generated from a second magnetic field generating unit, there are advantages in that it is easy to control the magnetic field and control a micro magnetic robot using the magnetic field.

In addition, since a magnetic field generating device moves automatically in accordance with the movement of the micro magnetic robot, there are advantages in that a procedure can become simpler and the burden on patients and medical staff can be reduced by shortening the procedure time.

In addition, there are advantages in that it is possible to prevent a malfunction of the micro magnetic robot due to the heat generation in a core and prevent damage to the device by effectively blocking the heat that may be generated in the core.

### DESCRIPTION OF DRAWINGS

FIG. 1 is a perspective view of one embodiment of a magnetic field generating device for a precision procedure according to the present invention.

FIG. 2 is an exploded perspective view of a driving unit applied to the magnetic field generating device for a precision procedure according to the present invention.

FIG. 3 is a schematic block diagram of a control module applied to the magnetic field generating device for a precision procedure according to the present invention.

FIGS. 4 to 6 are a perspective view, a longitudinal cross-sectional view, and a plan view of a first magnetic field generating unit and a second magnetic field generating unit applied to the magnetic field generating device for a precision procedure according to the present invention, respectively.

FIG. 7 is a set of pictures in place of drawings showing a magnetic field distribution of a magnetic field generating device to which the related art is applied and a magnetic field distribution of the magnetic field generating device for a precision procedure according to the present invention.

FIG. 8 is a set of side state views of a process of folding an upper coil in the magnetic field generating device for a precision procedure according to the present invention.

FIG. 9 shows an implementation view of a state in which the magnetic field generating device for a precision procedure according to the present invention is applied to a conventional imaging system.

### MODES OF THE INVENTION

Hereinafter, embodiments of the present invention will be described in detail with reference to the accompanying drawings so that those skilled in the art to which the present invention pertains can easily implement the present invention. However, it is not intended to limit the present invention to specific embodiments, and it should be understood that all changes, equivalents, or substitutes included in the spirit and technical scope of the present invention are included.

When an element is described as “connected” or “coupled” to another element, it should be understood that the element may be directly connected or coupled to the other element or other elements may be present therebetween.

On the other hand, when an element is described as “directly connected” or “directly coupled” to another element, it should be understood that there are no other elements therebetween.

The terms used in the present specification are merely used to describe specific embodiments and are not intended to limit the present invention. Singular expressions include plural expressions unless the context clearly indicates otherwise. In the present application, it should be understood that terms such as “comprise” or “have” are intended to specify the presence of features, numbers, processes, operations, elements, parts, or combinations thereof described in the specification and do not preclude the possibility of the presence or addition of one or more other features, numbers, processes, operations, elements, parts, or combinations thereof.

Unless otherwise defined, all terms used herein including technical or scientific terms have the same meanings as those generally understood by those skilled in the art to which the present invention pertains. Terms such as those defined in commonly used dictionaries should be interpreted as having meanings consistent with their meanings in the context of the relevant technology and are not to be interpreted in an ideal or excessively formal sense unless explicitly defined in the present application.

The term “module” described in the present specification refers to one unit for processing a specific function or operation, which may mean hardware or software, or a combination of hardware and software.

Terms or words used in the present specification and claims should not be construed as limited to commonly or dictionary meanings and should be construed as meanings and concepts consistent with the technical spirit of the present invention based on the principle that the inventors may appropriately define the concepts of terms to describe their invention in the best way. In addition, when there is no other definition of used technical terms and scientific terms, the terms have a meaning commonly understood by those skilled in the art to which the present invention pertains, and descriptions of known functions and configurations that may unnecessarily obscure the gist of the present invention will be omitted from the following description and accompanying drawings. The drawings introduced below are provided as examples in order to sufficiently convey the spirit of the present invention to those skilled in the art. Therefore, the present invention is not limited to the drawings presented below and may be embodied in other forms. In addition, throughout the specification, the same reference numerals denote the same elements. It should be noted that the same elements in drawings are denoted by the same reference numerals as much as possible in the drawings.

The present invention relates to a magnetic field generating device for a precision procedure, and more specifically, to a magnetic field generation for a precision procedure, which may safely and precisely move a procedural robot (hereinafter referred to as a “micro magnetic robot”) inserted into a patient's body to a target location using a magnetic field by extending the magnetic field generated from a magnetic coil upward to extend an area of the magnetic field.

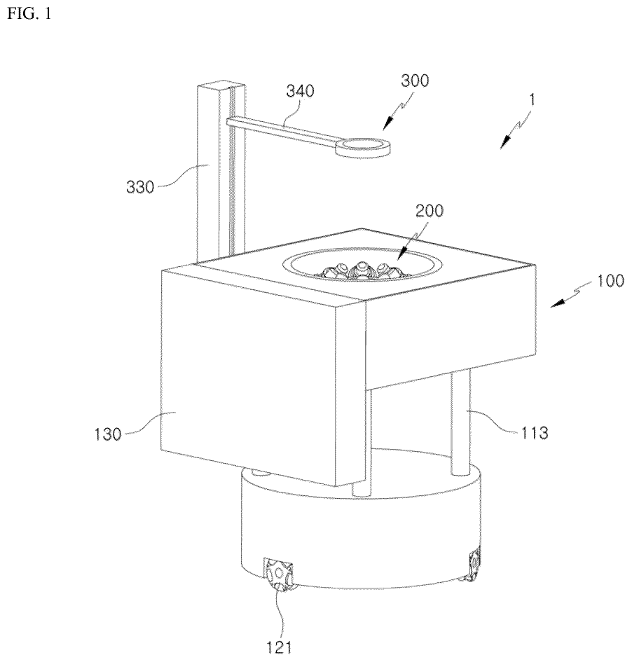

FIG. 1 is a perspective view of one embodiment of the magnetic field generating device for a precision procedure according to the present invention.

Referring to FIG. 1, the magnetic field generating device 1 for a precision procedure according to the present invention includes a moving unit 100, a first magnetic field generating unit 200, and a second magnetic field generating unit 300.

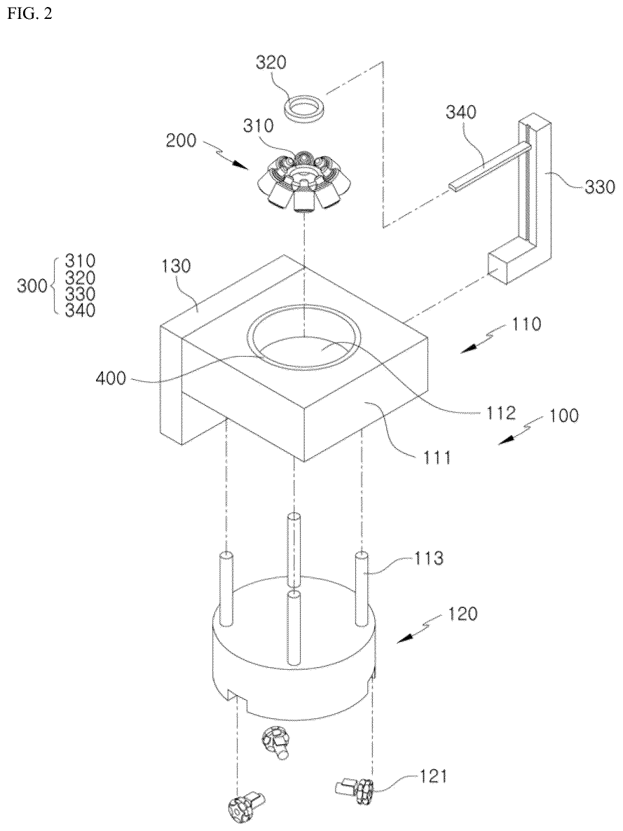

FIG. 2 is an exploded perspective view of a driving unit applied to the magnetic field generating device for a precision procedure according to the present invention.

Referring to FIG. 2, the moving unit 100 includes an accommodating case 110 in which the first magnetic field generating unit 200 is installed, a driving module 120 including driving wheels at a bottom portion to be movable, and a control module 130.

The accommodating case 110 includes a housing 111 having a predetermined shape and an accommodating recess 112 recessed from an upper portion to a lower portion of the housing 111, and the first magnetic field generating unit 200 is disposed and installed inside the accommodating recess 112.

The driving module 120 is installed under the accommodating case 110 to move the magnetic field generating device 1 for a precision procedure according to the present invention by driving and includes driving wheels 121 driven in contact with the bottom portion thereof.

The driving wheel 121 includes a driving motor and a wheel, and an omni wheel that may be driven in a rotation direction and a direction perpendicular to the rotation direction may be applied as the wheel.

In the above configuration, the accommodating case 110 and the driving module 120 are coupled through lifting bars 113.

The lifting bars 113 are configured to have a length adjusted through one device selected from the accommodating case 110 and the driving module 120, and by adjusting the lengths of the lifting bars 113, a height between the accommodating case 110 and the driving module 120 is adjusted. That is, the height of the accommodating case 110 is changed by adjusting the lengths of the lifting bars 113.

The control module 130 serves to provide a driving control signal to the driving module based on received location information and control power (current) applied to the first magnetic field generating unit 200 and the second magnetic field generating unit 300.

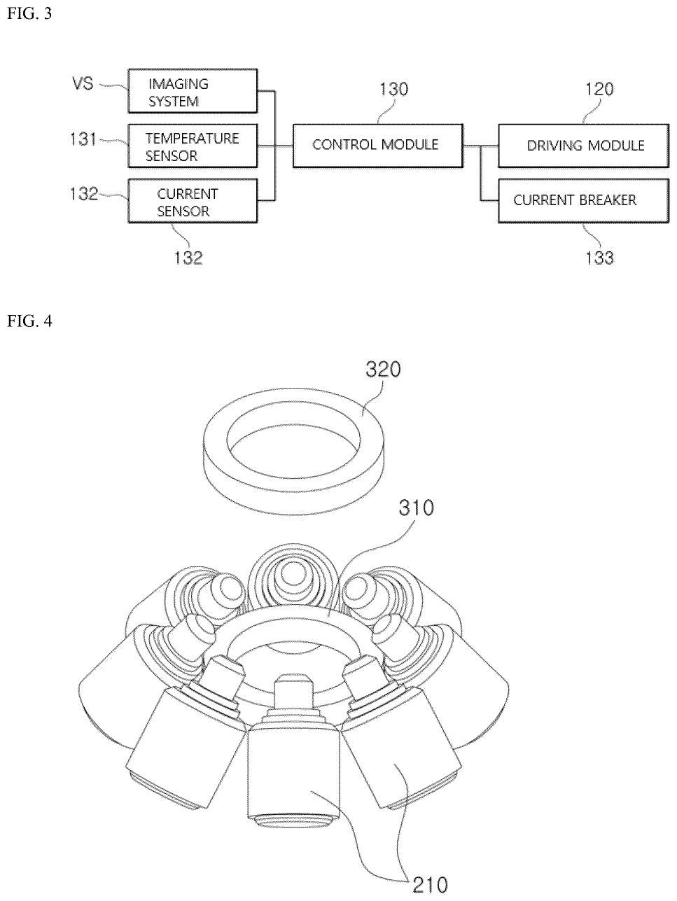

FIG. 3 shows a schematic configuration of the control module applied to the magnetic field generation for a precision procedure according to the present invention.

Referring to FIG. 3, the control module 130 receives image information transmitted from an external imaging system VS (e.g., X-rays) and detects a location of the micro magnetic robot based on the received image, and transmits the driving control signal to the driving module 120. Therefore, the driving module 120 moves to a controlled setting location by driving the driving wheels 121 according to the driving control signal.

In addition, the first magnetic field generating unit 200 and the second magnetic field generating unit 300 output different magnetic field intensities by the applied power (current).

By the applied power, the first magnetic field generating unit 200 and the second magnetic field generating unit 300 generate heat by a resistance element, and as a resistance value is changed by the generated heat, the required magnetic field intensity may not be satisfied. In addition, there is a problem that when heat is continuously generated, the device may be damaged due to burnout.

Therefore, the control module 130 may be configured to decrease the temperatures of the first magnetic field generating unit 200 and the second magnetic field generating unit 300 based on temperature values detected by a temperature sensor 131.

For example, the control module 130 is configured to detect a temperature value of the first magnetic field generating unit 200 or the second magnetic field generating unit 300 through the temperature sensor 131 and decrease the temperatures of the first magnetic field generating unit 200 and the second magnetic field generating unit 300 by one or more cooling methods selected from air cooling and water cooling when the detected temperature value exceeds a set first temperature value.

In addition, when the temperature value of the first magnetic field generating unit 200 or the second magnetic field generating unit 300 detected through the temperature sensor 131 exceeds a set second temperature value, the control module 130 is configured to prevent damage to the device due to burnout by blocking (interrupting) the current applied to the first magnetic field generating unit 200 or the second magnetic field generating unit 300.

That is, the control module 130 is configured to perform control such that the temperature of the first magnetic field generating unit 200 or the second magnetic field generating unit 300 is lowered when the temperature value detected by the temperature sensor 131 exceeds the first set value and is smaller than or equal to the second set value and to ensure the safety of the device by blocking the current when the temperature value detected by the temperature sensor 131 exceeds the second set value.

Alternatively, the method of decreasing the temperature of the first magnetic field generating unit 200 or the second magnetic field generating unit 300 may be driven by a self-cooling method rather than the control according to the temperature value detected by the temperature sensor 131. For example, it goes without saying that the control module 130 may be configured to be cooled by one or more cooling methods selected from air cooling and water cooling according to the operation of the first magnetic field generating unit 200.

In addition, the control module 130 includes a current sensor 132 and when the current applied to the first magnetic field generating unit 200 and the second magnetic field generating unit exceeds a set current value based on a current value detected by the current sensor 132, the control module 130 is configured to block the current applied to the first magnetic field generating unit 200 and the second magnetic field generating unit 300 to prevent fire and burnout due to overcurrent.

That is, when the current value detected by the current sensor 132 temporarily exceeds the set current value or continuously exceeds the set current value, the control module 130 is configured to block the power applied to the first magnetic field generating unit 200 or the second magnetic field generating unit 300 by outputting a trip signal to a current breaker 133 to block the current applied to the first magnetic field generating unit 200 or the second magnetic field generating unit 300.

The first magnetic field generating unit 200 generates a magnetic field by the applied power (current) and includes a plurality of magnetic coils 210.

The second magnetic field generating unit 300 generates a magnetic field by the applied power (current) and includes a lower coil 310 and an upper coil 320.

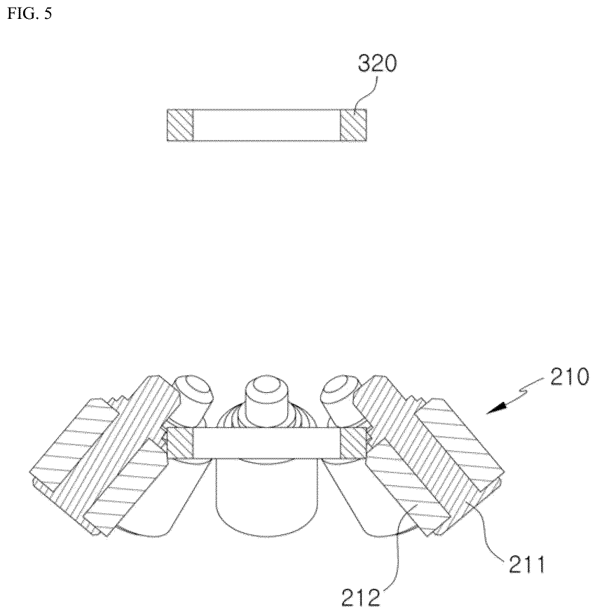

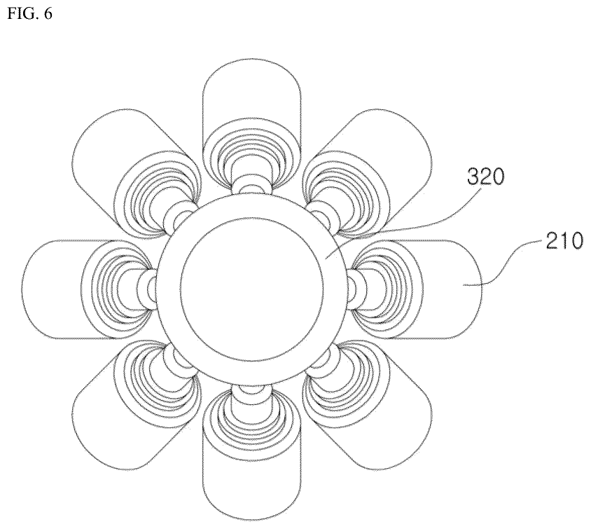

FIGS. 4 to 6 are a perspective view, a longitudinal cross-sectional view, and a plan view of the first magnetic field generating unit and the second magnetic field generating unit applied to the magnetic field generating device for a precision procedure according to the present invention, respectively.

The first magnetic field generating unit 200 includes the plurality of magnetic coils 210 arranged in a circle, and six to ten magnetic coils 210 are optionally installed. Preferably, eight magnetic coils 210 may be installed.

The magnetic coil 210 includes a core 211 and a coil 212 wound around the core 211.

Here, the magnetic coil 210 adopts an air-cooling method or a water-cooling method to decrease heat generation due to the application of current.

To apply the air-cooling method, a blower may be installed around the coil 211. To apply the water-cooling method, the coil 211 may have a hollow shaft and may be configured to allow coolant to flow through the hollow shaft.

The second magnetic field generating unit 300 generates a magnetic field to overlap a magnetic field area generated by the first magnetic field generating unit 200, the lower coil 310 is disposed inside the magnetic coils 210 of the first magnetic field generating unit 200, the upper coil 310 is disposed directly above the first magnetic field generating unit 200, and the micro magnetic robot to be controlled is located between the lower coil 310 and the upper coil 320.

In this case, the lower coil 310 and the upper coil 320 are formed in a donut shape and an imaging system (not shown) for detecting the micro magnetic robot is disposed above the upper coil 320.

That is, since the upper coil 320 is formed in a donut shape, it is possible to capture an image of the micro magnetic robot located inside a patient using the imaging system without interference from the upper coil 320.

Here, the lower coil 310 and the upper coil 320 may be formed of Helmholtz coils.

The Helmholtz coil is a device for generating a uniform magnetic field and is composed of two identical circular coils, and the two coils share a central axis with an effective area interposed therebetween and are located parallel to each other.

Meanwhile, a magnetic field shielding material 400 for shielding the magnetic field may be installed on an inner wall of the accommodating recess 112 (see FIG. 2).

The magnetic field shielding material has advantages of being able to minimize magnetic field interference from the surroundings and being able to generate a strong magnetic field by focusing the magnetic field.

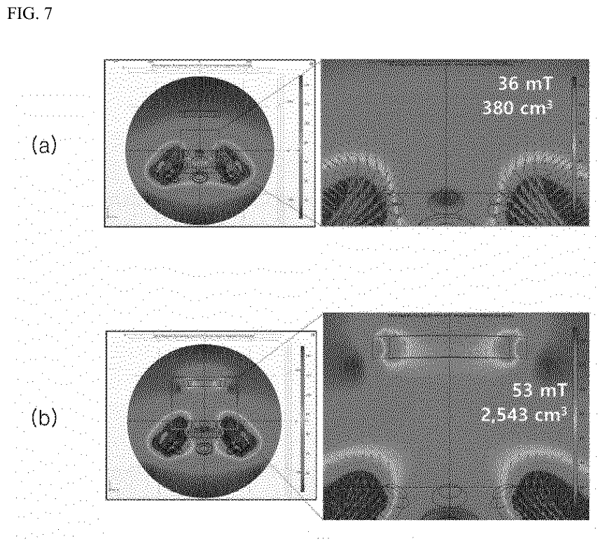

FIG. 7 is a set of pictures in place of drawings showing a magnetic field distribution of a magnetic field generating device to which the related art is applied and a magnetic field distribution of the magnetic field generating device for a precision procedure according to the present invention.

FIG. 7A shows the magnetic field distribution of the magnetic field generating device to which the related art is applied, and it can be seen that the generated magnetic field is concentrated near the generating device.

FIG. 7B shows the magnetic field distribution generated by the first magnetic field generating unit and the second magnetic field generating unit applied to the magnetic field generating device for a precision procedure according to the present invention, and shows that the generated magnetic field is extended and distributed upward.

Specifically, while the magnetic field intensity at a central portion thereof was 36 mT and a magnetic field control range was 380 cm³ in the case of the conventional magnetic field generating device, in the case of the magnetic field generating device for a precision procedure according to the present invention it was found that the magnetic field intensity at a central portion thereof was 53 mT and a magnetic field control range was 2,543 cm³. That is, the magnetic field intensity was simulated to increase by about 1.5 times, and the magnetic field control range was simulated to increase by about 6.7 times, which means that although the range in which the magnetic field is uniformly generated is wider, the magnetic field intensity may be relatively stronger compared to the related art.

As the magnetic field extends upward, the magnetic fields generated by the first magnetic field generating unit and the second magnetic field generating unit are concentrated on and distributed in an effective part, and a magnetic field suitable for controlling the micro magnetic robot may be generated.

That is, compared to the conventional magnetic field generating device, in the magnetic field generating device for a precision procedure according to the present invention, there is an advantage in that the magnetic field generated at the relatively upper side is extended and distributed, and the movement of the micro magnetic robot may be more easily controlled using the generated magnetic field.

Meanwhile, a separate support is required to allow the upper coil 320 to be spaced apart from and disposed directly above the first magnetic field generating unit 200 and the lower coil 310.

Referring to FIG. 2, the second magnetic field generating unit 300 further includes a vertical frame 330 installed upright on the moving unit 100 and a horizontal frame 340 disposed perpendicular to the vertical frame 330, and the upper coil 320 is installed on the horizontal frame 340.

Here, the horizontal frame 340 is configured to be vertically movable along the vertical frame 330. Therefore, since the horizontal frame 340 may move upward and downward along the vertical frame 330 to have a variable height, there is an advantage in that the horizontal frame 340 may be adjusted to a location suitable for the patient's body.

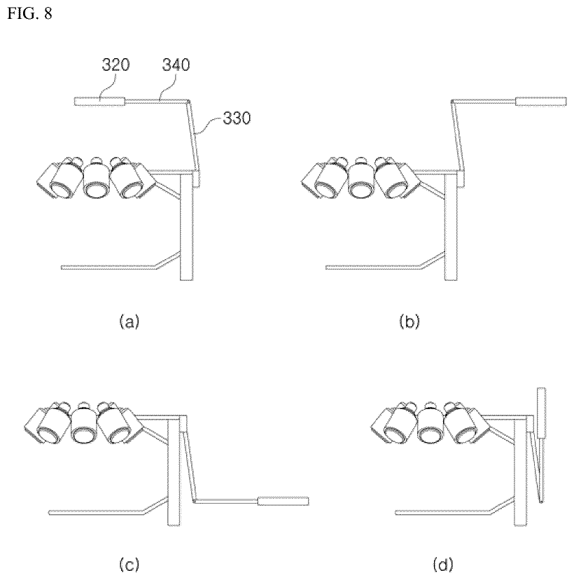

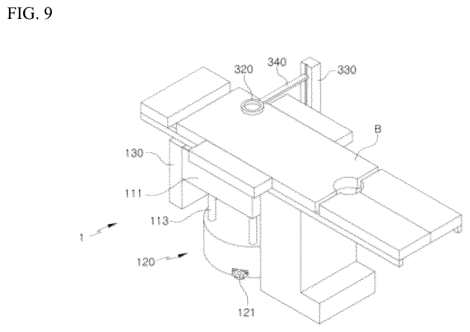

FIG. 8 is a set of side state views briefly showing a process of folding the upper coil in the magnetic field generating device for a precision procedure according to the present invention.

Referring to FIG. 8, in a use state in FIG. 8A, the lower coil 310 and the upper coil 320 are disposed to face each other. In this case, the vertical frame 330 and the horizontal frame 340 are coupled or connected by a hinge or a rotatable joint. Since known configurations may be applied as configurations of the hinge coupling and joint coupling, detailed descriptions and drawings of the configurations are omitted.

FIG. 8B is a side view of a state in which the vertical frame 330 is rotated 180° using the horizontal frame 340 as a rotational axis, and FIG. 8C is a side view of a state in which the vertical frame 330 is rotated 180° with respect to a connection contact point of the moving unit.

In addition, FIG. 8D is a side view of a state in which the horizontal frame 340 is rotated 90° upward with respect to a connection contact point of the vertical frame 330.

As described above, since the upper coil 320 is configured to be folded by rotation and folding, it is possible to achieve convenience in a process of storing or transporting the magnetic field generating device for a precision procedure according to the present invention.

FIG. 9 shows an implementation view of a state in which the magnetic field generating device for a precision procedure according to the present invention is applied to a conventional imaging system.

Referring to FIG. 9, a bed B on which a patient lies is disposed between the magnetic field generating device 1 for a precision procedure according to the present invention. Specifically, the bed B is disposed between the first magnetic field generating unit 200 and the upper coil 320 of the second magnetic field generating unit 300 of the magnetic field generating device 1 for a precision procedure. That is, the patient is located between the first magnetic field generating unit 200 and the upper coil 320 of the second magnetic field generating unit 300, and the magnetic fields generated from the first magnetic field generating unit 200 and the second magnetic field generating unit are distributed in a patient area.

According to the present invention, since the magnetic field generated from the first magnetic field generating unit is concentrated in the effective space by the magnetic field generated from the second magnetic field generating unit, there are advantages in that it is easy to control the magnetic field and control the micro magnetic robot using the magnetic field.

In addition, since the magnetic field generating device moves automatically in accordance with the movement of the micro magnetic robot, there are advantages in that a procedure can become simpler and the burden on patients and medical staff can be reduced by shortening the procedure time.

In addition, there are advantages in that it is possible to prevent a malfunction of the micro magnetic robot due to the heat generation in the core and prevent damage to the device by effectively blocking the heat that may be generated in the core.

Although the present invention has been described above with reference to embodiments of the present invention, those skilled in the art will understand that the present invention can be modified and changed in various ways without departing from the spirit and scope of the present invention as specified in the appended claims.

This invention was supported by the TIPS R&D (50%). Daegu Special Zone (30%). TIPS Startup Commercialization (10%), and TIPS Overseas Marketing (10%) projects.

1. A magnetic field generating device for a precision procedure, comprising:
a moving unit configured to be movable;
a first magnetic field generating unit installed on the moving unit; and
a second magnetic field generating unit configured to generate a magnetic field to overlap a magnetic field area generated by the first magnetic field generating unit.

2. The magnetic field generating device of claim 1, wherein the moving unit includes:
an accommodating case in which the first magnetic field generating unit is installed;
a driving module driven according to a driving control signal; and
a control module configured to provide the driving control signal to the driving module based on received location information.

3. The magnetic field generating device of claim 2, wherein the control module detects a temperature value and applied current value of the first magnetic field generating unit or the second magnetic field generating unit and controls to interrupt the current applied to the first magnetic field generating unit or the second magnetic field generating unit on the detected temperature value and current value.

4. The magnetic field generating device of claim 1, wherein the first magnetic field generating unit is composed of a magnetic coil including:
a core; and
a coil wound around the core.

5. The magnetic field generating device of claim 4, wherein the core is configured to be cooled by one or more cooling methods selected from air cooling and water cooling.

6. The magnetic field generating device of claim 4, wherein the magnetic coil is configured as a plurality of magnetic coils at predetermined intervals.

7. The magnetic field generating device of claim 4, wherein a magnetic field shielding material is applied to a frame of the magnetic coil to focus the magnetic field.

8. The magnetic field generating device of claim 1, wherein the second magnetic field generating unit includes:
a lower coil disposed under the first magnetic field generating unit; and
an upper coil disposed above the first magnetic field generating unit, and
the lower coil and the upper coil are each formed in a donut shape.

9. The magnetic field generating device of claim 8, comprising:
a vertical frame installed upright on the moving unit; and
a horizontal frame disposed perpendicular to the vertical frame,
wherein the upper coil is installed on the horizontal frame.

10. The magnetic field generating device of claim 9, wherein the horizontal frame is configured to be vertically movable along the vertical frame.

11. The magnetic field generating device of claim 1, wherein the second magnetic field generating unit is formed of a Helmholtz coil.
